(19) United States
(12) Patent Application Publication
(10) Pub. No.: US 2019/0287672 A1
(43) Pub. Date: Sep. 19, 2019

(54) ARCHIVE SERVER AND SYSTEM

(71) Applicant: Camilo Alejandro Molina, Baltimore, MD (US)

(72) Inventor: Camilo Alejandro Molina, Baltimore, MD (US)

(21) Appl. No.: 16/351,398

(22) Filed: Mar. 12, 2019

Related U.S. Application Data
(60) Provisional application No. 62/644,985, filed on Mar. 19, 2018.

(51) Int. Cl. G16H 30/20; H04L 29/08

(52) U.S. Cl. CPC G16H 30/20; H04L 67/2804

(57) ABSTRACT

Embodiments of the invention provide a server including a processor coupled to a server storage configured to be coupled to a network, and a non-transitory memory in communication with the processor and storing logic instructions. When executed by the processor, the logic instructions cause the server to operate a gateway manager establishing a dataflow between a user and the server storage based on an access right, and processing requests for a data coupling from an application running at a networked device associated to upload or download medical data to or from the server storage. Further, applying process tools operating on the medical data, and providing a rule-based matching between two or more users based on the medical data and a rule, where medical data associated with a first user is made accessible to another user based on the medical data and a rule.

### RELATED APPLICATIONS

This application claims priority to United States provisional patent application No. 62/644,985, filed on Mar. 19, 2018, the entire contents of which are incorporated herein by reference.

### BACKGROUND

Modern medical imaging data is typically collected at the request of a credentialed healthcare provider (e.g., such as a primary care physician) via imaging devices such as, but not limited to, single or multi-planar fluoroscopy, computed axial tomography (“CAT”), magnetic resonance imaging (“MM”), ultrasonography, positron emission tomography (“PET”), or single photon emission computed tomography (“SPECT”). Modern medical imaging is data dense and usually packaged in large data files that present a significant obstacle to the secure and efficient dissemination of medical imaging within different networks and institutions.

The imaging data is typically acquired at a medical establishment, processed, stored and subsequently made available for interpretation to a qualified professional or medical provider, such as a radiologist, who interprets the images and reports findings and a diagnosis. The diagnostic report is then made available to both the original requesting healthcare provider and the patient. The requesting healthcare provider can manage the care if it is within their scope of practice, or provide a referral to a subspecialty provider (e.g., a neurosurgeon or other specialist) for further management of the diagnosed condition. The imaged patient can be given a digital or printed copy of acquired medical imaging and accompanying medical imaging interpretation report, as provided by a credentialed radiologist.

Most medical institutions employ client-server versus cloud-based picture archiving systems (PACS) for medical image uploads, storage, and distribution. Providers that do not have rights to access medical records within an internal institutional local or cloud network are prevented from accessing, viewing, or processing medical images and related information in the network. The distribution of medical imaging to providers outside internal client or cloud-based networks can occur physically using printed slides, CD-ROM, or other physical media. Medical imaging data can also be sent to providers outside a network via electronic means, such as, but not limited to, e-mail or manual file sharing cloud-based systems.

Some institutions have improved upon this process by establishing institutional upload servers where clients (e.g., patients, providers, insurance agents) can upload medical imaging data for review by professional or medical providers within said institution. The data within these servers is secure and available only to providers within the institution who have access to that institution's network. Patients do not currently have a single central system for upload, storage, processing and distribution of medical imaging to providers across different institutions with varying network access credentials.

Cloud-based medical imaging systems are well known to provide cost and workflow advantages in comparison to older systems. A cloud data and processing system can refer to any system in which data storage and computational processing functions are performed by one or more remote server-based systems. Allocating data storage and computational processing to remote cloud-based servers decreases end-user workstation hardware and software requirements. References to “thin” versus “thick” clients are made in reference to partition of resources between end-user client and the cloud-based systems. Cloud-based systems partition data storage and computational processes between the end-user client station and cloud-based server. When the cloud-based server performs the majority of data storage and computational process functions, the system is referred to a “thin client” application. In contrast, when the functions of the system are predominantly performed by the end-user client, such a system is referred to as a “thick client” application. As such, end-user workstations can run thin clients such as a web browser or a mobile tablet or phone application to access functions performed by the higher capacity cloud-based server system. Further, end-users accessing cloud-based applications through a thin client are not required to install and/or troubleshoot software or hardware on access workstations. Importantly, cloud-based systems can be accessed through wide area networks (WAN, i.e. the internet) permitting access by multiple users in varying locations with varying thin clients. Furthermore, components of the cloud-based system can reside anywhere and be managed by multiple third parties. Thus, cloud-based systems have been widely implemented to handle medical imaging storage and processing.

Modern medical imaging cloud systems face major challenges. As medical image acquisition modalities become more advanced to provide higher quality images with increased spatial resolution, the file sizes become increasingly large. For example, a standard brain MM study can yield over 1000 images with uncompressed file sizes ranging from 50 MB to 9 GB depending on varying acquisition factors. Storage and post-acquisition processing of such large file sizes inherently places high resource burdens on both local and cloud-based imaging systems. In order to decrease the burden of storing and processing large medical imaging file sizes, conventional cloud-based imaging systems use a variety of methods to decrease file size. Such methods can include file compression to smaller file formats, image re-scaling or resizing, systematic addition or subtraction of pixel information referred to those familiar in the art as interpolation, and manual or batched processed data cropping in which the image corner margins are removed to decrease file size. Given the sensitivity in maintaining privacy of transmitted medical data, extra-ordinary security measures are implemented throughout such systems, thereby limiting access and collaboration to users across different intuitions. Hence, due to the high technical resources needed to operate these cloud-based medical imaging systems, institutions who apply these solutions must still allocate significant financial resources to their implementation, maintenance, and security.

Financial models for implementation of cloud-based imaging services are varied. For example, third party providers of cloud-based solutions can license-out services based on the number of users, and study uploads and downloads with a per study fee varying according to volume of images or image data in said study. Licensing can also occur according to number of studies viewed or processed, including a range of costs according to image processing requirements. Licensing costs also often factor the user type, the study purpose (e.g., clinical versus research utility), allotted bandwidth, medical study billing codes, the number of users with access to the cloud-based system and/or the number of times the system is accessed by such users.

Given the relative inflated cost of implementing, maintaining, and using cloud-based imaging systems for individual medical institutions, centralized vendor-neutral archives are not widely implemented. A true “vendor-neutral” archive (“VNA”) gives medical institutions the ability to send images to any cloud-based system from any PACS. This means that a true VNA would permit access to centralized storage and processing of medical images to practitioners across any institution in any location.

Although sound in theory, true VNAs are rare because permitting universal access of image upload and download to such a system would be very costly and resource intensive. In such a system, where all medical institutions have universal access, it becomes unclear how to spread the financial burden required for the operation of such a large system. Thus, the majority of cloud-based imaging systems are currently institution specific with limited integration across different institutions. The current unavailability of a centralized VNA, where any patient or practitioner can access the cloud-based system to upload, process, download, and grant access to any practitioner in any location, limits patient access to healthcare practitioners and/or other medical services.

In conventional cloud-based imaging systems, an administrator can grant radiologists and radiology technicians the ability to employ all image processing, rendering and editing rights possible by the system; however, other users such as patients or non-radiologists only have the right to view certain image data with minimal ability to process or edit images. Accordingly, conventional cloud-based systems have access control systems that assign user privileges based on user professional or medical credentials or lack thereof. For example, as in U.S. Pat. No. 8,682,049B2, a cloud server can receive a request for accessing medical image data from a user over a network, where the cloud server provides image processing services to a plurality of users using a plurality of image processing tools provided by the cloud server. The cloud server determines user privileges of the users for accessing the medical image data, where the user privileges are related to the storage, upload, download, and manipulation of the medical imaging data. Thus, the availability of the image processing tools is limited to defined user privileges according to user type.

### SUMMARY

Some embodiments include a server comprising a processor coupled to a server storage configured to be coupled to a network, and a non-transitory computer readable memory in communication with the processor and storing logic instructions. In some embodiments, when executed by the processor, the logic instructions can cause the server to operate a gateway manager coupled to the server storage over the network, where the gateway manager establishes a data transfer between at least one user and the server storage based on an access right. In some embodiments, when executed by the processor, the logic instructions can cause the server to process a request for data from an application running at a networked device associated with the user. In some embodiments, when executed by the processor, the logic instructions can cause the server to act on a request from the user to upload or download medical data to or from the server storage. In some embodiments, when executed by the processor, the logic instructions can cause the server to act on a request from the user to access and apply one or more process tools operating on at least a portion of the medical data stored on the server storage according to input from a user. In some embodiments, when executed by the processor, the logic instructions can cause the server to provide a rule-based matching between two or more users based on at least a portion of the medical data and at least one rule, where at least some medical data associated with a first user is made accessible to at least one other user based on the at least one rule.

Some embodiments comprise the non-transitory computer readable memory storing logic instructions that, when executed by the processor, can cause the server to anonymize at least a portion of the medical data uploaded to the server storage. In some further embodiments, the server anonymizing at least a portion of the medical data automatically relabels the at least a portion of the medical data with non-identifying user characteristics.

Some embodiments further comprise the non-transitory computer readable memory storing logic instructions that, when executed by the processor, can cause the server to image process at least a portion of the medical data using the one or more process tools operating on at least a portion of the medical data stored on the storage. In some embodiments, the image processing comprises at least one of rescaling, margin cropping, compression, file size reduction, processing metadata to a searchable list and/or index, encryption, modality conversion, sharing of medical data between two or more users based on the at least one rule.

In some embodiments, the medical data comprises medical image data. In some further embodiments, the medical image data comprises radiological study data. In some embodiments, the access right is governed by a license. In other embodiments, the access right is license-free. In some embodiments, the server storage is a cloud-based server storage coupled to the network.

In some embodiments of the invention, the at least one other user is a medical practitioner or healthcare service provider and the first user is a patient or potential patient of the at least one other user. In some embodiments of the invention, the at least one rule comprises a preference of the at least one other user. In some further embodiments, the at least one rule comprises a preference of the at least one other user selected from one or more parameters or inputs on a display of the system that is coupled to the server storage.

In some embodiments, the at least one rule relates to the type of professional or medical degree of the at least one other user, educational and clinical training history of the at least one other user, professional or medical certifications of the at least one other user, subspecialty certifications of the at least one other user, institutional or hospital credentials of the at least one other user's scope of practice, a background check of the at least one other user, medical license information of the at least one other user, and clinical interests within scope of practice of the at least one other user. In some embodiments, the one or more parameters or inputs comprises a diagnosis, a self-reported diagnosis, a study type, insurance status, geographic distance between the first user and the at least one other user, the age of the first user, and/or study date.

In some embodiments, the non-transitory computer readable memory storing logic instructions that, when executed by the processor, can cause the server to analyze clinical relevancy of at least a portion of the medical data using the one or more process tools operating on at least a portion of the medical data stored on the storage, server identifying any non-clinically relevant medical data and any clinically relevant medical data based on one or more internal rules; and further configured to optionally discard non-relevant medical data.

In some further embodiments, the non-transitory computer readable memory storing logic instructions that, when executed by the processor, can cause the server to match at some of the medical data based on a diagnosis defined by at least one of the users and/or extracted from metadata from the medical data or from data associated with stored medical data. In some other embodiments, the non-transitory computer readable memory storing logic instructions that, when executed by the processor, can cause the server to upload the medical data directly from an institution or facility where the medical data was acquired using an upload link and temporary access key provided by a user.

In some embodiments of the invention, the non-transitory computer readable memory storing logic instructions that, when executed by the processor, can cause the server to enable the first user to view engagements offered by the at least one other user after the at least one other user has reviewed at least some of the medical data associated with the first user. In some further embodiments, the non-transitory computer readable memory storing logic instructions that, when executed by the processor, can cause the server to enable the first user to receive an access key for an engagement offered by the at least one other user after the at least one other user has reviewed at least some of the medical data associated with the first user, and further to enable the first user to book an appointment with the at least one other user through the server using the access key.

### DESCRIPTION OF THE DRAWINGS

Embodiments of the invention are illustrated by way of example and not by way of limitation in the accompanying drawings in which associated reference numerals refer to similar elements.

FIG. 1 shows a block diagram illustrating cloud-based user data and image processing systems according to some embodiments of the invention.

FIG. 2 shows a diagram illustrating examples of server processing functions and associated network connectivity according to some embodiments of the invention.

FIG. 3 illustrates a flow structure diagram illustrating a workflow method for consumer user data upload, download, and professional or medical user engagement in accordance with some embodiments of the invention.

FIG. 4 shows a flow diagram illustrating a workflow method for professional or medical user data collection yielding corresponding consumer user matches, review of matched consumer imaging, and direct consumer clinical engagement in accordance with some embodiments of the invention.

FIG. 5 illustrates a graphical user interface (GUI) of a consumer user workstation upload application in accordance with some embodiments of the invention.

FIG. 6 depicts a GUI of a consumer user workstation showing downloads of imaging studies in native or post processing modalities including 3D volume renditions in accordance with some embodiments of the invention.

FIG. 7 depicts a professional or medical user workstation GUI for entering and selecting matching preferences in accordance with some embodiments of the invention.

FIG. 8 depicts a professional or medical user workstation GUI for selecting and viewing anonymous user imaging studies in native or post processing modalities including 3D volume renditions in accordance with some embodiments of the invention.

FIG. 9A shows a workflow for consumer user review and acceptance of offered clinical engagements in accordance with some embodiments of the invention.

FIG. 9B shows a GUI for consumer user review and acceptance of offered clinical engagements in accordance with some embodiments of the invention.

FIG. 10A shows a workflow for professional or medical user clinical engagement of matched consumer user images in accordance with some embodiments of the invention.

FIG. 10B shows a GUI for professional or medical user clinical engagement of matched consumer user images in accordance with some embodiments of the invention.

FIG. 11 is a block diagram illustrating an example of automatic data cropping and image processing according to consumer user input in accordance with some embodiments of the invention.

FIG. 12 is a flow diagram illustrating consumer user account creation process in accordance with some embodiments of the invention.

FIG. 13 is a flow diagram illustrating an independent consumer account payment collection system in accordance with some embodiments of the invention.

FIG. 14 is a flow diagram illustrating professional or medical user account creation process in accordance with some embodiments of the invention.

FIG. 15 is a flow diagram illustrating the smart data cropping algorithm process in accordance with some embodiments of the invention.

FIG. 16 illustrates a computer system for processing or operating one or more systems or methods of FIGS. 1-8, 9A-9B, 10A-10B, and 11-15 in accordance with some embodiments of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives that fall within the scope of embodiments of the invention.

Various embodiments and aspects of the invention are described with reference to details in subsequent discussion, with the accompanying figures illustrating drawings representing various embodiments of the invention. Numerous specific details are described to provide a thorough comprehension of certain embodiments of the invention. In certain circumstances, conventional knowledge in the art is not elaborated to facilitate concise description and discussion of certain embodiments of the invention.

Reference to “some embodiments”, “an embodiment”, “certain embodiments”, “some embodiments”, etc., within the following discussion means that a certain structure, feature, characteristic, or function described in association with an embodiment can be included in one or more embodiments of the invention. The reference to “some embodiments” or “an embodiment” throughout description of the invention does not necessarily refer to the same embodiment.

Some embodiments of the invention relate to cloud-based data and imaging processing systems permitting direct consumer user image processing and connection to a professional or medical user independent of the professional or medical user's location or institutional affiliation. Some embodiments include a cloud-based anonymous vendor-neutral archive (hereinafter “VNA”) for imaging-based direct matching of healthcare consumer to one or more professional healthcare practitioners or service providers.

According to some embodiments of the invention, the consumer user (e.g., such as a patient) can access a cloud-based system to: 1. enter anonymous demographic data and/or can 2. upload anonymous and unprocessed medical imaging data acquired independently. In some embodiments, collected demographic data excludes identifying information and imaging, and is anonymized via automatic removal of identifying internal imaging metadata. In some embodiments of the invention, a consumer user is provided with de-identified processed imaging for view in modality of consumer user choice. In some embodiments, a professional or medical user (e.g., a physician) can access a cloud-based system and enter specific practice data such as location, specialty, and clinical interests. In some embodiments, a cloud-based system can process data entered by consumer and professional or medical users, and provide a corresponding match and direct connection of a consumer user to a professional or medical user. In some embodiments, the professional or medical user can subsequently review criteria matched de-identified consumer user medical imaging information, and engage one or more consumer users directly through at least one internal engagement system.

Some embodiments include a cloud-based imaging system that can provide advanced image and data processing functions. In at least one non-limiting embodiment, a cloud-based imaging system can be any combination of a server, data store, firewall, router, and/or gateway management system. According to some embodiments, the described invention can comprise one or more cloud-based systems.

In some embodiments of the invention, the cloud-based imaging system can include a cloud server that can be configured to provide data and imaging processing services to users (e.g., including, but not limited to consumer users, or educational users, professional or medical users of any affiliation in any location). As defined herein, for the purposes of description, a “consumer user” is broadly defined as an individual (e.g., such as a patient) in possession of or able to provide personal medical imaging that is independently acquired and interpreted prior to accessing or interacting with any embodiments of the invention described herein. Further, as defined herein, for the purposes of description, a “professional user” is broadly defined as any licensed medical healthcare practitioner or service provider in the United States (e.g., such as an MD, DO, or DDS), or corresponding qualifications in any respective country accessing or interacting with any embodiment of the invention described herein. For example, as used herein, a professional user can be a physician, surgeon, dentist, veterinarian, or other medical service provider. As used herein, a “consumer” can be a patient or person seeking medical advice or treatment. In other embodiments, the “consumer” can be a patient or person seeking education. In some embodiments, the “consumer” can be a medical or healthcare provider, or other professional user. Further, as defined herein, for the purposes of description, an “educational user” is broadly defined as any individual or group accessing or interacting with any embodiment of the invention described herein with educational or academic purposes such as in research.

In some embodiments, any cloud server can also be referred to as a data, medical data or medical imaging processing server with the capability of processing medical data (e.g., such as one or more medical images) that enables or processes user data anonymization, encryption, data compression, data cropping, and modality conversion including, but not limited to, automated matching or sharing of images with selected users, and any combination thereof. In another embodiment of the invention, a cloud server can also be referred to as a user matching server where consumer and professional or medical users are matched according to a set of rules. In another embodiment of the invention, a cloud server can be referred to as a portal for educational user access to anonymous imaging data banks and image processing tools. In some inventions, a cloud server can match medical data (e.g., such as medical imaging data) based on a diagnosis defined by a user and/or extracted from metadata or from data associated with stored medical data.

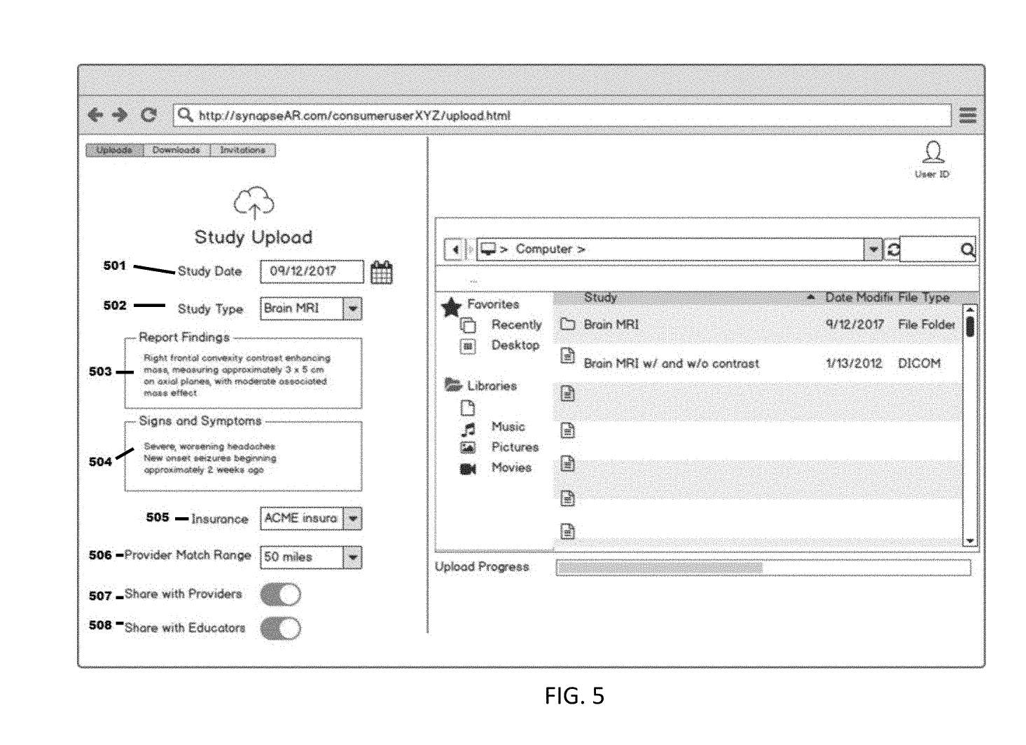

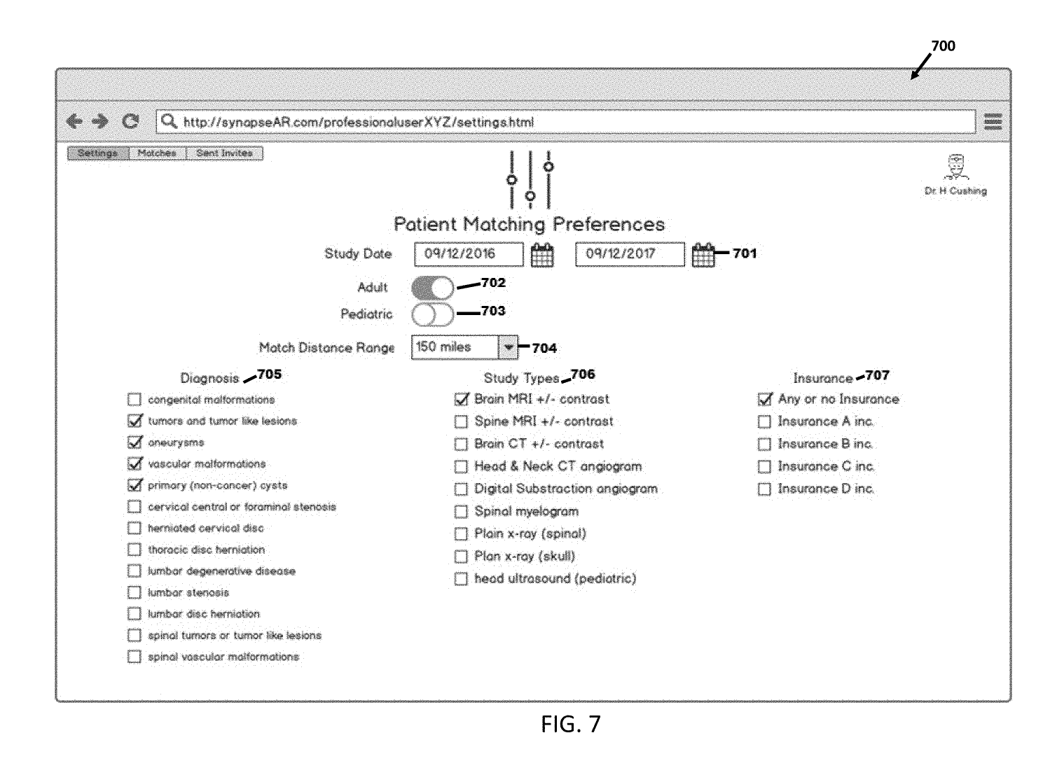

In some embodiments of the invention, the set of rules for matching consumer users to professional or medical users can be an “all or none system”, where all of the following variables must match to elicit a positive match. In other embodiments of the invention, the set of rules for matching consumer users to professional or medical users can be one or more of the following variables. For example, in some embodiments, the variables can be consumer user uploaded study date (see 501 in FIG. 5) matching study date range preference entered by professional or medical user. (see date 701 of FIG. 7).

Further, in some embodiments, the variables can comprise a consumer user age dichotomized as adult (greater than or equal to 18 years old) or pediatric (less than 18 years old) matching adult (702 of FIG. 7) and/or pediatric (703 of FIG. 7) professional or medical user matching preference.

Further, in some embodiments, the variables can comprise a consumer user geographical range preference (506 of FIG. 5) matching professional or medical user geographical range preference (704 of FIG. 7).

Further, in some embodiments, the variables can comprise a consumer user study type (502 of FIG. 5) being among study types selected by professional or medical user (706 of FIG. 7).

Further, in some embodiments, the variables can comprise a consumer user insurance status (505 of FIG. 5) professional or medical user insurance acceptance preferences (707 of FIG. 7).

Further, in some embodiments, the variables can comprise a consumer user's self-reported diagnosis (503 of FIG. 5 with signs and symptoms 504) matching professional or medical users' clinical scope of practice (705 of FIG. 7).

According to some embodiments, the cloud-based imaging system of any embodiments described herein can include a data gateway management system that is coupled to a network and one or more cloud systems or one or more cloud components such as servers or data storage systems. Any network of any embodiments described herein can be a local area network (“LAN”), metropolitan area network (“MAN”), or a wide area network (“WAN”) such as intranet or internet. Some embodiments can use more than one type of network. For example, some embodiments can use a LAN and WAN, or a MAN and WAN, and so on. In some embodiments, the data gateway manager can comprise any combination of a router, server, or data storage. In some embodiments, the data gateway manager can be configured to automatically and/or manually transfer data between users (consumer, educational, or professional or medical users) according to an administrator defined set of user roles and privileges. In some embodiments, the data gateway manager can be configured to transmit data automatically based on user data matching with one or more users being recipients of said data. For example, in some embodiments, a consumer user whose imaging and demographics match preference criteria of one or more professional or medical users, can have imaging automatically transmitted to said professional or medical users independent of that professional or medical user's institutional affiliation and location. In another embodiment of the invention, the data gateway manager can be configured to transmit data manually, with examples including, but not limited to, manual consumer user upload of anonymized medical imaging to server, manual consumer user input of user characteristics such as demographics and diagnosis, and manual professional or medical user download and review of anonymous matched consumer user's imaging. In some embodiments of the invention, the gateway manager can upload on a consumer's behalf any anonymized medical imaging directly from the medical institution that acquired the imaging.

According to some embodiments, the cloud-based system can include a data store or data storage system. In some further embodiments, the data storage system can comprise any combination of a gateway manager, router, server, data storage hardware, and/or data storage software. In some embodiments, the data storage system can be contained within one or more storage systems within a local network of some embodiments or in a secure external facility such as a Microsoft® relational database management systems (“RDBMS”) e.g., Microsoft® SQL Server. Microsoft® is a registered trademark of Microsoft Inc., Redmond, Wash.

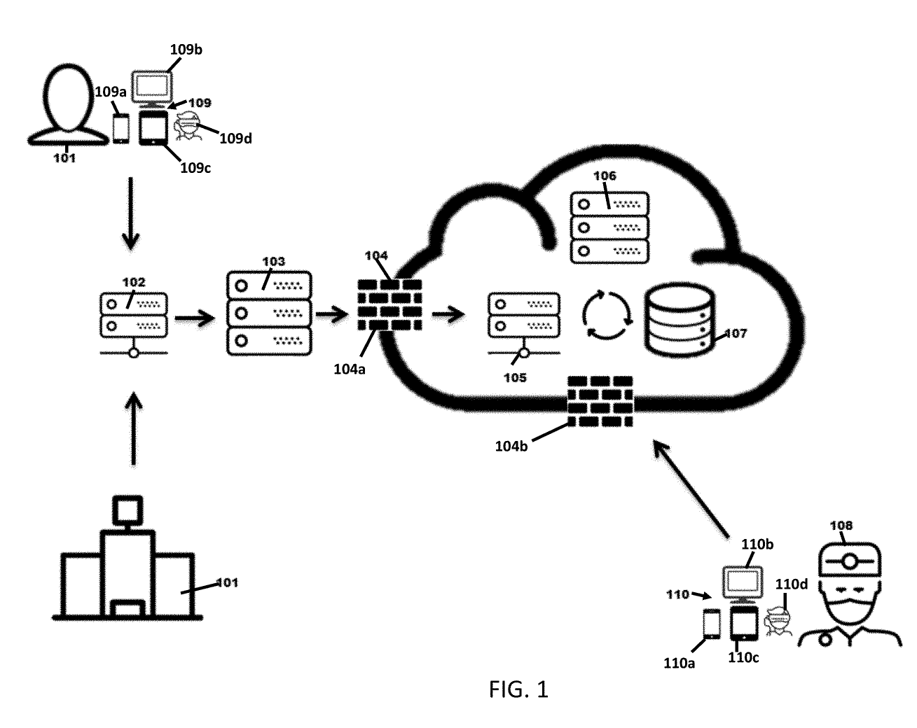

FIG. 1 is a block diagram illustrating cloud-based user data and image processing systems containing a data gateway manager according to certain embodiments of the invention. In some embodiments, server 103 and server 106 are illustrated as separate, but can be one or a plurality of servers functioning in series or parallel for performance of specific processes. In some embodiments, gateway managers 102 and 105 are illustrated as separate data gateway managers but can be one or plural gateway managers functioning in series or parallel for performance of specific processes. In some embodiments, all servers, gateway management systems, or data management systems described within varying embodiments can be self-contained within one or more internal cloud systems, or partitioned to exist among external secure networks maintained by third-party hosts. In some embodiments, as illustrated in FIG. 1, consumer users 101 can make upload requests to the data gateway manager 102 for access to the server 103. In some embodiments, the server 103 can function to anonymize uploaded imaging data by automatically removing internal imaging metadata. In some embodiments, the server 103 can function to anonymize uploaded imaging data by automatically re-labeling uploaded imaging with non-identifying user characteristics (e.g., consumer user ID and consumer user demographics).

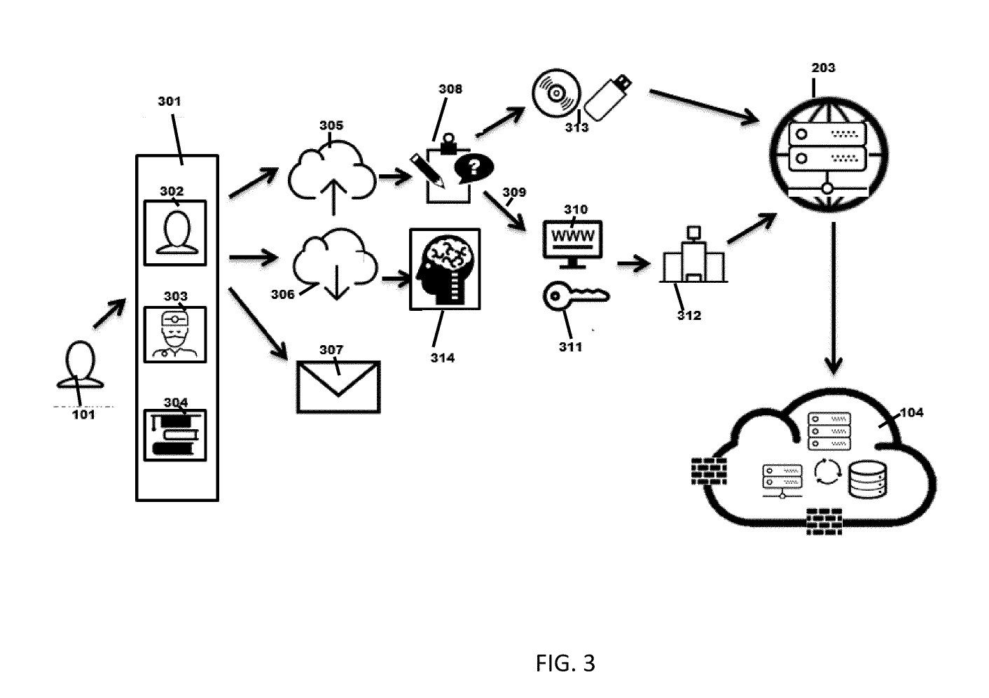

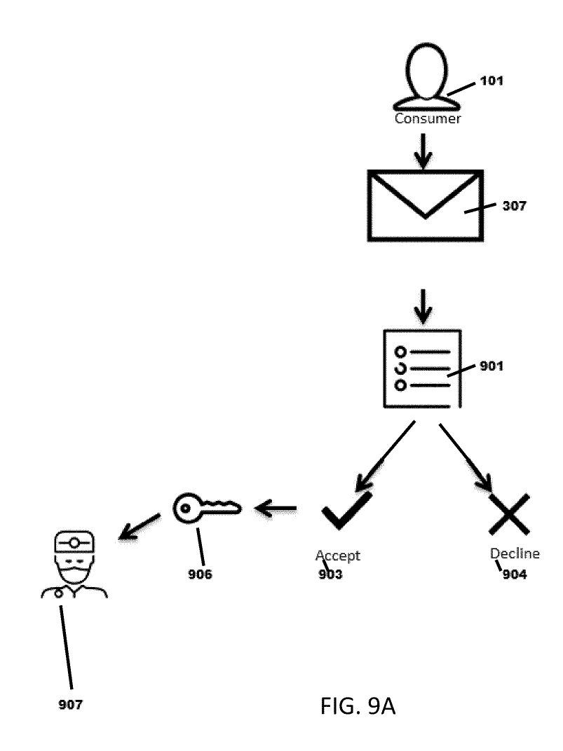

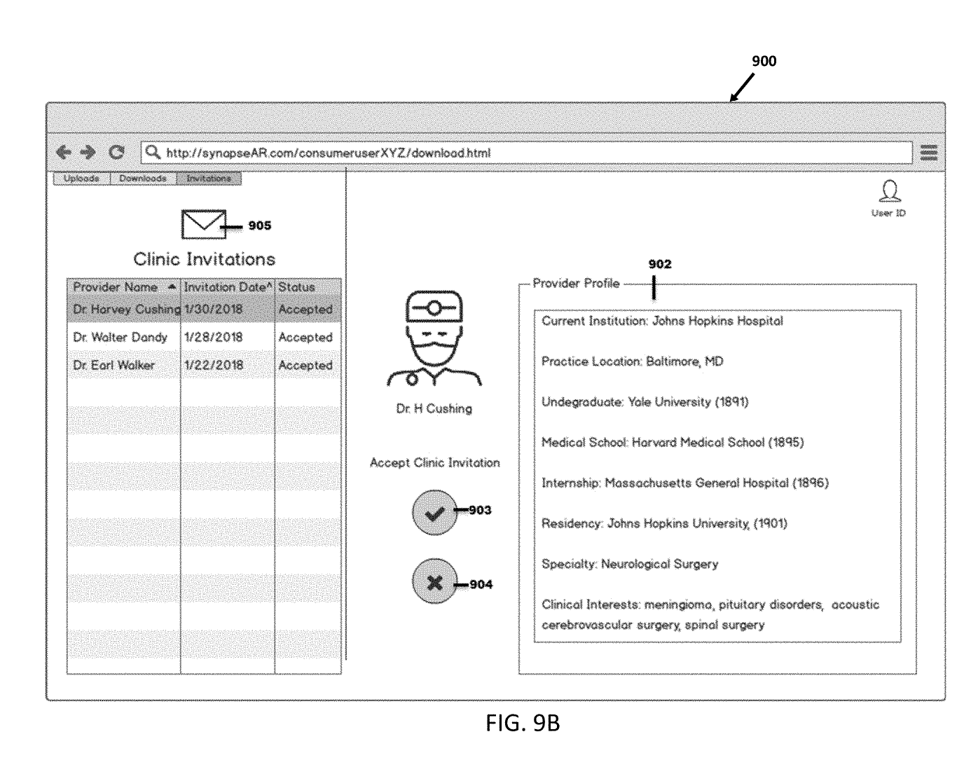

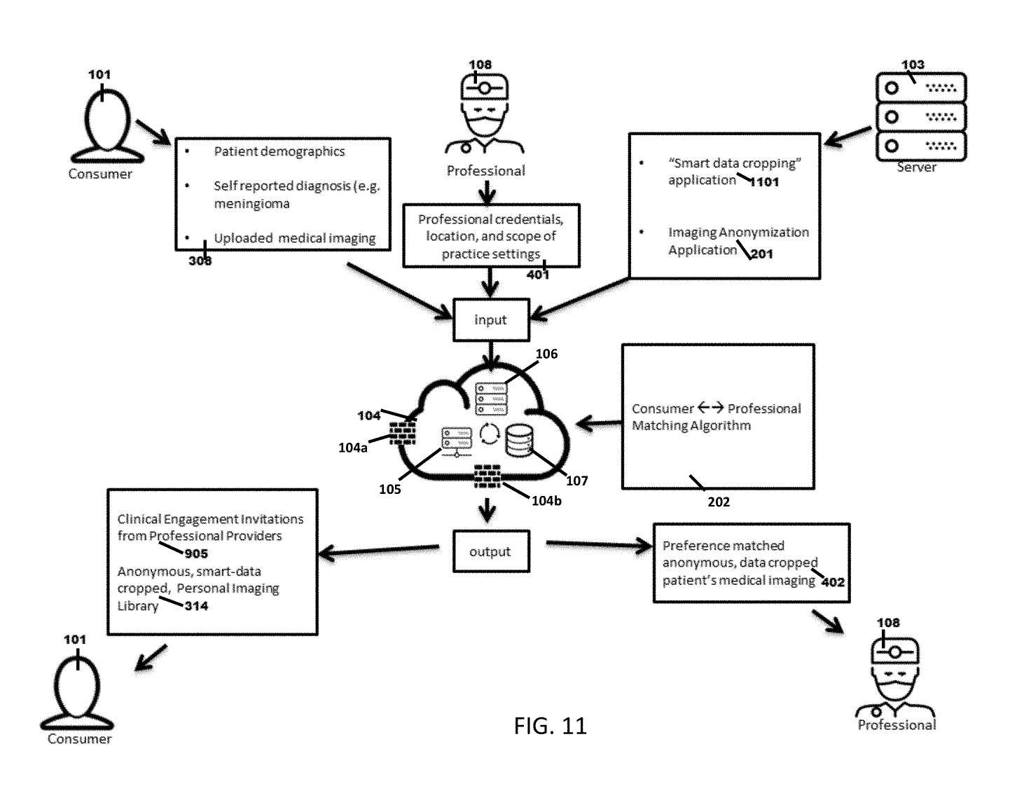

Some embodiments include automatic data cropping and image processing according to user input. In some embodiments, the consumer user 101 can provide input 308 (see FIG. 3), including, but not limited to, demographic information, and/or self-reported diagnosis information, and/or uploaded medical imaging, that in some embodiments, can be provided on storage media 313. In some embodiments, input from a professional or medical user 108 can include professional or medical credentials, location, and scope of practice settings (matching preferences 401). Referring to the flow diagram of FIG. 11, in some embodiments, the server 103 can function as a smart data cropping system (1101 of FIG. 11). In some embodiments, the smart data cropping system 1101 can function to automatically filter and select “clinically relevant” imaging data. Further, in some embodiments, the smart data cropping system 1101 can optionally discard remaining non-relevant clinical data to make the uploaded imaging file smaller, and thereby decreasing data transfer and storage resource requirements of the present cloud-based system. For example, in some embodiments, a patient uploading radiological study data such as a brain MRI for a user-inputted diagnosis of meningioma can result in only a certain sequence and series (i.e. only certain images) of the MRI being transmitted, with the remainder of the image data discarded. In some embodiments, the determination of what imaging data (e.g., series or sequences) are clinically relevant is based upon one or more internal rules executed by the system. In some embodiments, output can comprise engagements offered by professional or medical users who have reviewed a particular consumer user's data and subsequently offered the consumer user 101 an opportunity to engage in a personal clinical setting (represented as 905, FIG. 9B), and anonymous, smart-data cropped, personal imaging library 314 (FIG. 3).

Referring to FIG. 1, in some embodiments, the gateway manager 105 can receive an update from server 103 that new de-identified, encrypted, compressed, and/or cropped imaging data is available for transmission to server 106. In some embodiments, server 106 can function to execute server applications such as communication with data storage system 107, and provide an access control system, and advanced imaging processing including, but not limited to, 3D volume reconstructions, and imaging data matching to user preferences and characteristics.

In some embodiments of the invention, the gateway manager 105 can receive an update that server 106 has executed requested imaging processing functions, and can transmit an update to consumer user 101 that anonymized processed imaging is available for download in multiple file formats compatible with multiple thin clients 109. In some embodiments, clients 109 can comprise one or more of a mobile phone 109a, computer system 109b, tablet or personal digital assistant 109c, and/or wearable computer system 109d.

In some embodiments, professional or medical users can request access to server 106 through gateway manager 105 to download de-identified user imaging and demographic data that matches professional or medical scope of practice and clinical interests. Further, in some embodiments, both consumer and professional or medical users 101, 108 can be enabled with access to a cloud-based system through multiple consumer 109 and/or professional or medical thin clients 110. In some embodiments, thin clients have the advantage of offloading resource intensive invention components such as complex image processing and data storage to various components of the present cloud-based system. In some embodiments, the medical or medical thin clients 110 can comprise one or more of a mobile phone 110a, computer system 110b, tablet or personal digital assistant 110c, and/or wearable computer system 110d.

Some embodiments include cloud-based imaging systems with smart data cropping functions employed in cloud-based imaging systems where a decrease in file size decreases imaging data storage, imaging processing, and transmission bandwidth resource needs. In some embodiments, most unprocessed medical images are encoded according to the “Digital Imaging and Communications in Medicine” (“DICOM”) standard. DICOM is a medical imaging standard created to integrate the many different existing medical imaging acquisition modalities. In some embodiments, DICOM files are composed of separate header and image data sets within the same file, and the header contains identifying information pertaining to the patient (i.e. patient identity and demographics) and the study (i.e. study type, imaging dimensions, color space, matrix size, and pixel intensity data). Pixel intensity data is integral to the structure of the file as it dictates the shade of gray for each individual pixel with as many as 65,536 possible shades of gray for each pixel. The respective shade of gray is encoded by 1 or 2 bytes of data for each gray pixel, or 3 bytes of data for color pixels. Hence image size is directly proportional to pixel density. For example, as gray image physical size doubles (128 by 128 pixels to 256×256 pixels), the corresponding encoding data size quadruples (16,384 versus 65,536 pixels).

In some embodiments of the invention, smart data cropping application 1101 contained within server 103 can receive and process uploaded de-identified consumer user imaging studies from consumer user 101. In some embodiments, data can be subsequently cropped by conventional methods such as re-scaling, margin cropping, and/or compression. In addition, in some embodiments, the data can be cropped by a method not previously described, referred to as smart data cropping, an algorithm within smart data cropping application 1101 of server 103, as illustrated in FIG. 11. In some embodiments, the smart data cropping application can function by storing, processing, and transmitting the imaging series that are of relevance to a particular consumer user's medical condition. For example, in some embodiments, for a consumer client that uploads a full series of radiological study data such as brain MRI data for a newly diagnosed convexity meningioma to server 103, the “smart data cropping” application can store, process, and transmit T1 axial and coronal post contrast imaging series to server 106, while discarding the remainder images (thereby significantly reducing the file size).

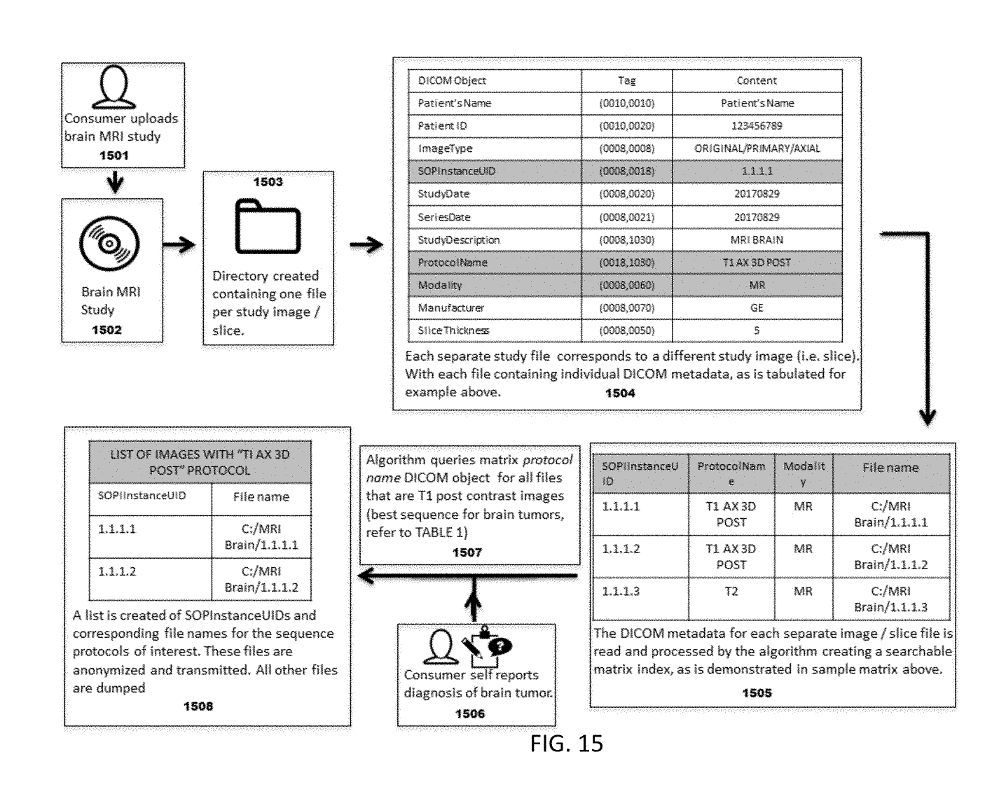

FIG. 15 is a flow diagram illustrating the smart data cropping process. In some embodiments, the smart data crop process described above can function via extracting the metadata specific for the type of study and study sequence for each separate study series. In some embodiments, a radiological study such as a brain MRI study 1502 is uploaded by a consumer (step 1501), and a directory 1503 is created with files 1504. In some embodiments, the DICOM metadata contains identifying information pertaining to the patient (i.e. patient identity and demographics) and the study (e.g., study type, study sequence, study series, imaging dimensions, color space, matrix size, and pixel intensity data). In some embodiments, in step 1507, an algorithm reads the DICOM file metadata, creating a searchable list matrix pairing DICOM metadata of interest to individual image file names. In some embodiments, the latter can create an index that can be searched (index 1505). In some embodiments, the consumer user can self-report the diagnosis via free text natural language input. In another embodiment, the consumer user can self-report the diagnosis via a pre-populated list (e.g., drop-down menu) (see step 1506).

The Table 1 (shown below) is an example of reference key employed by smart data cropping algorithm to select the best study and sequence for a particular diagnosis. In some embodiments, the self-reported diagnosis is cross referenced against a key (Table 1) where in the best imaging study sequence and series is identified for the respective entered consumer user diagnosis 1506. In some embodiments, the function can then search the index 1505 described above for the appropriate imaging study sequence and series. In some embodiments, the latter index search can return a list of file names corresponding to individual images that match the sequences and series of interest 1508. In some embodiments, these files can be extracted and processed for anonymization and transmission to cloud-based imaging system 104 that includes one or more firewalls 104a, 104b. In some embodiments, the remaining files can be discarded.

| | Best Imaging Study and Sequence |
|---|---|
| Cranial Pathology Diagnosis | |
| Intra-axial brain lesion | AXIAL T1 POST CONTRAST MR |
| Glioblastoma multiforme | AXIAL T1 POST CONTRAST MR |
| Low grade astrocytoma | AXIAL T2 NON-CONTRAST |
| | FLAIR MR |
| Metastatic brain lesion | AXIAL T1 POST CONTRAST MR |
| Meningioma | AXIAL T1 POST CONTRAST MR |
| Intracranial aneurysm | 4 VESSEL DIAGNOSTIC |
| | CEREBRAL ANGIOGRAM |
| Cavernous Malformation | AXIAL T2 NON-CONTRAST MR |
| Brain Abscess | AXIAL T1 POST CONTRAST & |
| | DIFFUSION RESTRICTION MR |
| Spinal Pathology Diagnosis | |
| Lumbar spondylolisthesis | SAGITTAL NON-CONTRAST |
| | LUMBAR COMPUTED TOMOGRAPHY |
| Herniated cervical, thoracic, | SAGITTAL T2 NON-CONTRAST |
| or lumbar disc | |
| Sagittal or coronal spinal | ANTERO-POSTERIOR AND |
| deformity | LATERAL STANDING SPINE |
| | PLAIN FILMS |
| Epidural or osseous spinal | SAGITTAL T1 POST CONTRAST |
| lesion | SPINAL MR |
| Intradural spinal lesion | SAGITTAL T1 POST CONTRAST |
| | SPINAL MR |
| Synovial cyst | AXIAL T2 NON-CONTRAST |
| | SPINAL MR |
| Spinal abscess | SAGITTAL T1 POST CONTRAST |
| | SPINAL MR |

Table 1 is an example of reference key or set of rules employed by smart data cropping algorithm to select the best study and sequence for a particular diagnosis.

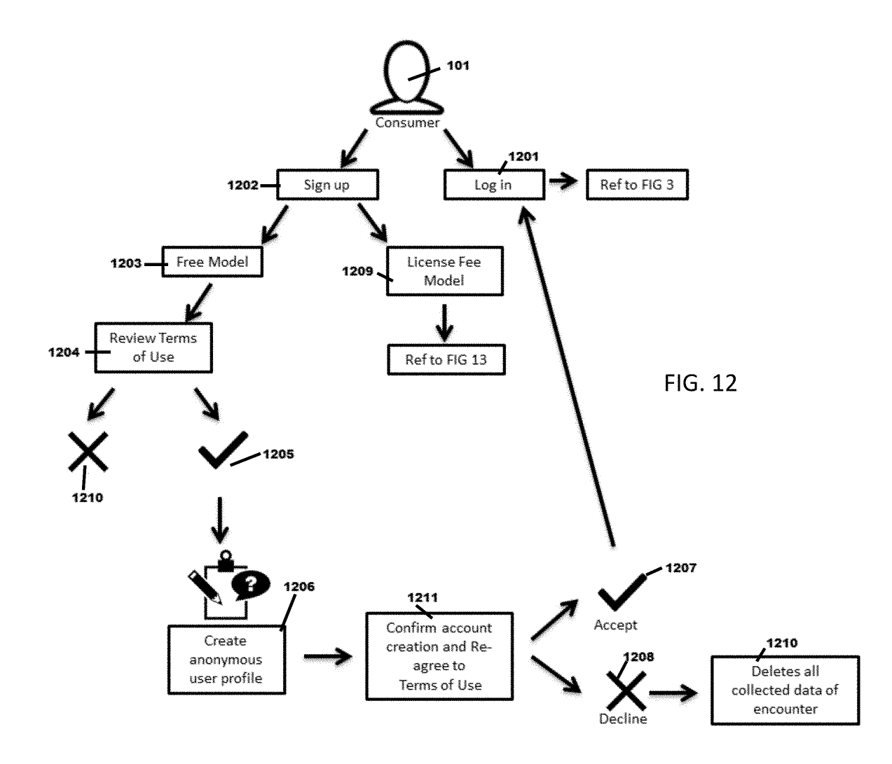

Referring to FIG. 12, in some embodiments, a new consumer user 101 can create an account for a cloud-based system access via sign up portal 1202. In other embodiments, the user 101 can log-on 1201 (see further in FIG. 3). In some embodiments, this access can be granted free of payment with no personal identifying information collected or stored within the data collection system (free model 1203). In this instance, users can be asked to create profiles with non-identifying user names, demographics, and non-specific past medical history data, and thus no identifying payment data is collected (step 1206). In some embodiments, prior to successfully creating a consumer user account, consumer users must accept a license and terms of use agreement twice; once before entering data to create account (step 1204, accept 1205) and once after entering requested data (accept 1207, step 1211, or decline 1208). In some embodiments, successful acceptance of both license and terms of use agreements can grant consumer user access to patient workspace platform 301 (FIG. 3). In some embodiments, selecting decline of license and terms of use agreements can result in deletion of all data stored within cloud-based system servers in association with failed account creation event (fail step 1210). Importantly, the aforementioned license and terms of use agreement, as it pertains to protect user safety and privacy, can include, but not be limited to, the following provisions and terms:

1). The provided cloud-based service is meant only as an anonymous screening method to efficiently and directly connect patients to professional or medical experts. Therefore, the system will not necessarily provide any direct diagnosis or therapeutic treatment service. In some embodiments, the cloud-based system can provide a mechanism for matching and facilitating direct off-line clinical engagement between a consumer user and a professional or medical user.

2). The consumer user's expressed understanding that one or more embodiments of the system do not guarantee a time frame for a positive match between a consumer and professional or medical user. Furthermore, the system does not make any claims or guidelines about the timeframe in which the direct engagement of consumer and professional or medical can occur following a positive match as identified-by the system.

3). As there is no timeframe for matching and engagement of consumer and professional or medical users, and the system is primarily intended for second opinions, consumer users are reminded to seek medical care from a qualified provider prior to system access. The term second opinion is used consistently with common healthcare jargon a patient seeking the opinion of multiple medical practitioners prior to deciding upon a diagnostic workup and treatment plan as offered by different experts.

4). A consumer user accessing this service can be reminded within this agreement, and on every login, to call emergency services for any acute medical condition and/or to immediately contact consumer user's primary care provider or emergency services if the user is uncertain regarding what constitutes a medical condition requiring prompt attention.

5). A consumer user's expressed understanding that the cloud-based system can reduce uploaded, transmitted, processed, and stored file sizes by “smart data cropping”. “Smart data cropping” in reference to the embodiment's application that functions to upload, transmit, process, and/or store certain parts of a study. Certain parts of the study (i.e. only certain images) refers to those images that are clinically relevant to consumer user's self-reported condition.

6). The automatic systems' “smart data cropping” application function is programmed upon clinical expertise, and thus a reflection of clinical best practices, and thereby may not represent the images that other clinicians would deem clinically relevant or irrelevant for a particular condition.

7). Referring to Table 1, the set of rules by which the “smart data cropping” application functions can be reprogrammed at any time upon decision of cloud-based system's administrators designated to manage and maintain the “smart data cropping” application.

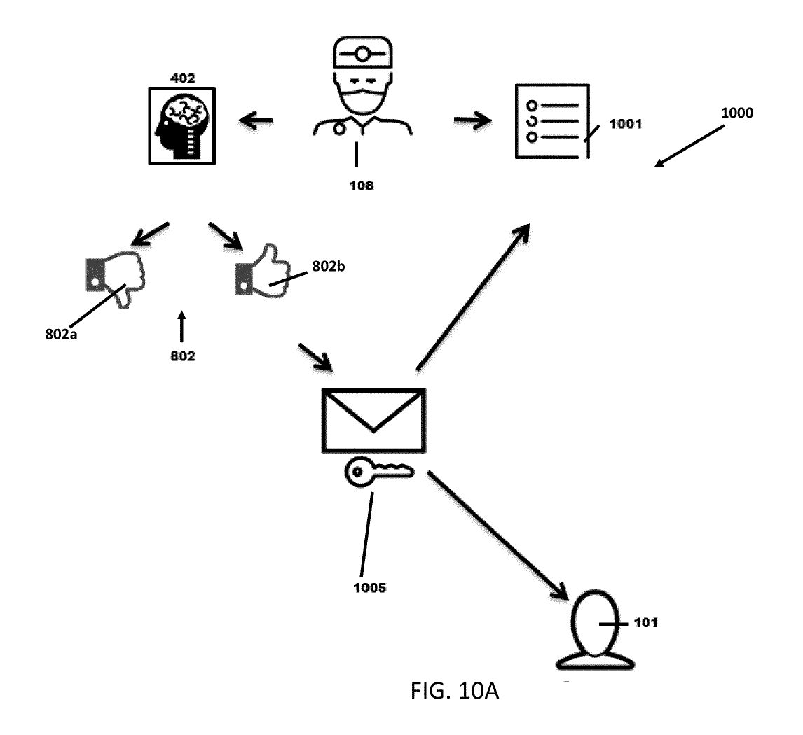

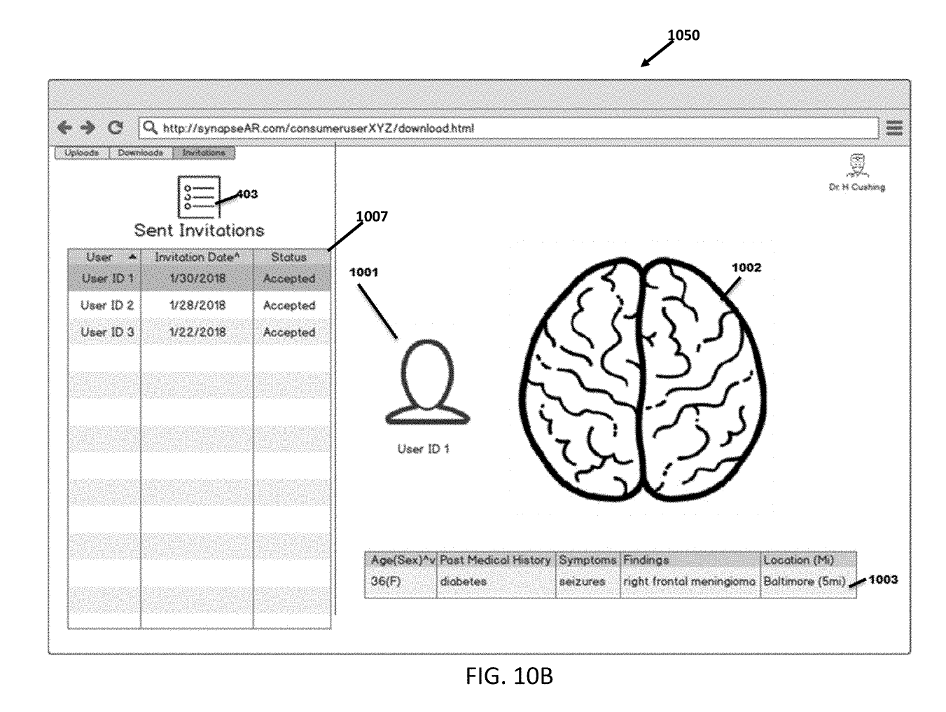

8). Professional or medical service providers or users are not permitted to freely communicate with consumer users through system, with the exception of the communication mechanisms provided within the cloud-based system clinical engagement platforms as illustrated in FIGS. 9A and 10A.

9). There is no identifying information maintained within the cloud-based system. All uploaded studies are anonymized upon immediate access to our cloud-based system.

10). Personal communication mediums or handles are also not collected or stored by system. In other words, identifying communication handles such as, but not limited to, name, physical address, phone numbers, messaging handles, and email address of consumer users are not collected.

11). Accounts for which login information is not available to a consumer user for whatever reason are deemed irretrievable, and can be automatically deleted.

12). Accounts with more than three incorrect login attempts are permanently deleted to protect user privacy.

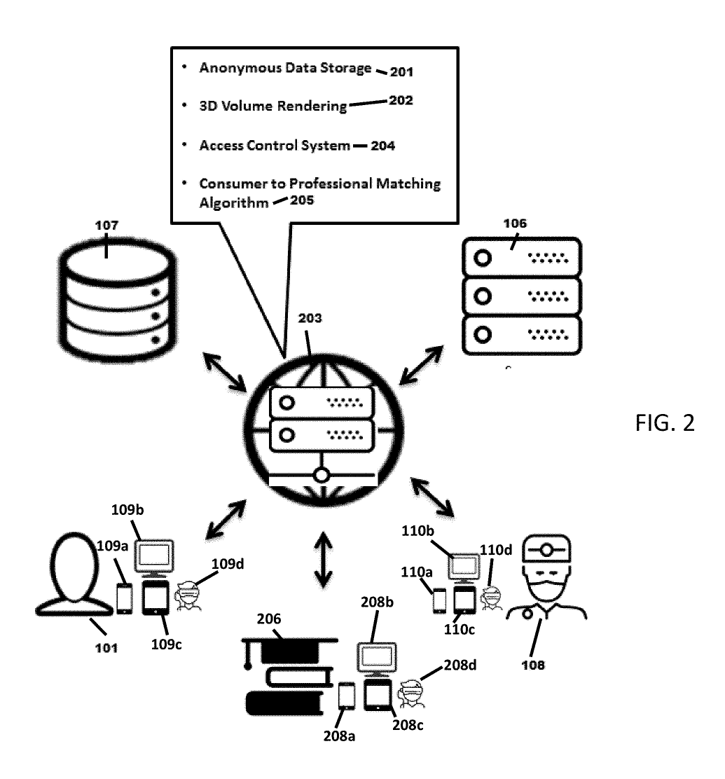

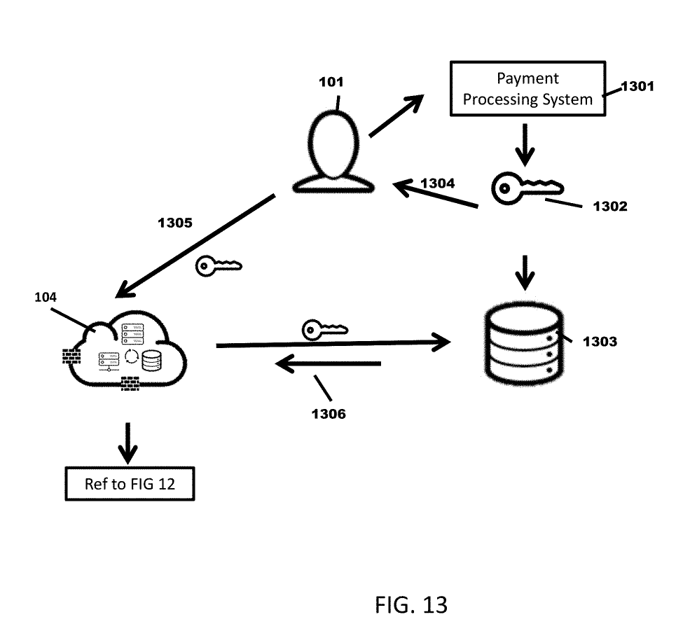

Referring to FIG. 13, in some embodiments, a consumer user account access to a patient platform (301of FIG. 3) can require payment. In such embodiments, any collection of identifying information with regard to payment data for access to cloud-based services and associated applications can be collected through a separate, and possibly third party, payment collection system that generates an encrypted random-access key to create a user profile (of payment processing system 1301 in FIG. 13). In some embodiments, a random-access key generator 1302 can be programmed to generate a key upon confirmation of payment for each consumer user. In some embodiments, the key can contain no identifying information. In some embodiments, upon generation of the key by application 1302, a copy of the key can be stored within the encrypted key storage application 1303, which can be located within data storage system 107 (FIG. 2). In some embodiments, another copy of the access key can be given to a consumer user (represented as arrow 1304). In some embodiments, upon that consumer user's entering of the key into the payment module (represented as arrow 1305, and license fee model 1209 of FIG. 12), the validity of the key can be confirmed by the encrypted key storage application 1303, 1306. In some embodiments, with verification of the key, the encrypted key storage application 1303 can delete all copies of existence of the key within the cloud-based system.

In some embodiments, if access keys generated by encrypted key generator 1302 are not used within a certain time frame of initial key generation, such keys can be permanently deleted, and the consumer user can forfeit a fee to access the system. In some embodiments, the consumer users can be notified of this clause prior to agreeing to payment submission.

As in U.S. Pat. No. 7,095,850B1, a random-access key updating method can efficiently generate one or more future keys in any order. The latter provides an encryption method and apparatus that provides forward secrecy, by updating the key using a one-way function after each encryption. In some embodiments, the by providing forward secrecy within a cipher, rather than through a key management system, forward secrecy can be added to cryptographic systems and protocols by using the cipher within an existing framework.

Referring to FIG. 3, according to some embodiments, a user 101 can access patient portal 301, and choosing the menu option “patient” 302 can lead a consumer user to choose among uploading a new study 305, downloading a prior uploaded imaging study 306, or viewing professional or medical user engagements 307. FIGS. 5, 6, and 9B illustrate a graphical user interface (hereinafter “GUI”) enabling consumer user access to different applications of cloud-based system via patient portal 301. According to some embodiments, a consumer user 101 can have a certain amount of storage space for storing anonymous uploaded studies, as determined by the cloud-based system's administrators. In some embodiments, allotted consumer user storage space capacity can be proportional to different licensing and/or payment methods. Referring to FIG. 5, showing a GUI illustration of consumer user workstation upload thin client in a desktop version, according to some embodiments, choosing an option to upload a new study 305 can direct a consumer user 101 to enter data such as, but not limited to, location, age, sex, past medical history, current signs and symptoms, type of imaging study being uploaded, imaging impression as reported on the professional or medical radiology report accompanying the uploaded image, confirmation that data can be anonymously shared with matching providers, and an option to share an anonymous study with a cloud-based system's educational image data bank. In some embodiments, following completion of the requested demographic and self-reported condition data, a consumer user 101 can be directed to upload the image via the thin client of their choice. In some embodiments, data can be selected to be shared with a provider 507 and/or an educator 508.

Referring to FIG. 3, according to some embodiments, a consumer user 101 can choose to have image data uploaded to the system directly from an institution or facility 312 where medical imaging was acquired. In some embodiments, a consumer user 101 can choose upload option 309, yielding a personal upload link 310 and temporary access key 311 to the institution or facility 312. In some embodiments, a consumer user 101 can subsequently provide the aforementioned upload link and temporary access key to the institution or facility where medical imaging was acquired. In some embodiments, the medical institution or facility can subsequently upload the study via that link and accompanying access key credentials. In some embodiments, the uploaded study can be automatically anonymized and added to consumer user's library of uploaded studies 306. In some embodiments, randomly generated keys can expire after a certain time frame.

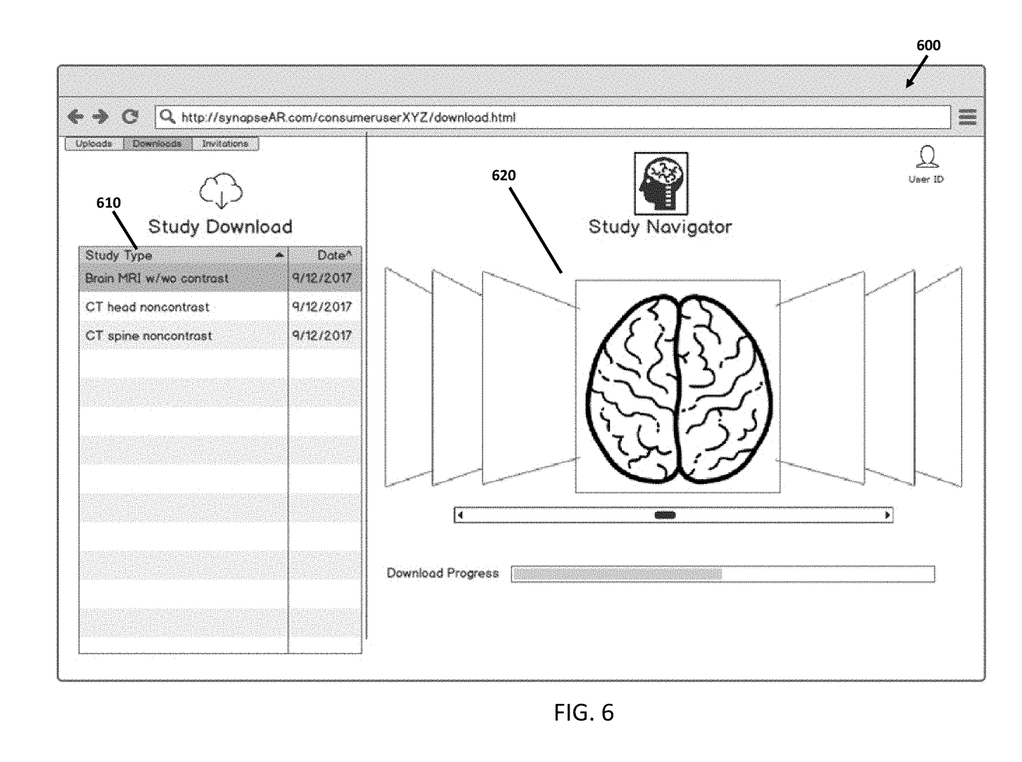

Referring to FIGS. 3 and 6, in some embodiments, when the consumer user 101 chooses an option to download prior uploaded studies 306, the consumer user 101 can be directed to choose among a list of prior uploaded studies to view. In some embodiments, a consumer user 101 can be enabled to choose among a number of different viewing modalities including, but not limited to, 3D volume reconstructions of prior uploaded imaging.

In some embodiments, professional or medical service providers or users can receive a list of matching clinical imaging studies for review by user matching system 205 (represented in FIG. 2), contained within server 106 (discussed earlier with respect to FIG. 1). Referring to FIG. 3, in some embodiments, a selection by a consumer user 101 to view professional or medical user engagements 307 can direct the consumer user 101 to a GUI, as illustrated in FIG. 9B (GUI 900), that lists the engagements offered by professional or medical service providers or users who have reviewed a particular consumer user's data, provides a view of a provider's profile 902, and subsequently can offer a consumer user 101 an opportunity to engage in a personal clinical setting (represented as 905).

Referring to FIG. 9A, in some embodiments, a consumer user 101 who has received a clinical engagement invitation (represented as 307) from professional or medical service providers or users can receive the aforementioned invitation in the form of randomly generated engagement validation access keys by random key generator 1302 (shown in FIG. 13). In some embodiments, engagements of menu option 901 can be accepted 903 or declined (904), and an access key can be used by a consumer user 101 when booking an appointment with a professional or medical user's place of practice (represented as 907) by phone, email, or electronic interface. For example, in some embodiments, a patient that receives a clinic invitation by a provider can receive an access key 906. In some embodiments, the patient can subsequently call the provider's office and inform a clinic administrator that they have been pre-screened for a clinic visit. In some embodiments, the patient can then provide the access key 906 to office staff of the professional or medical user's place of practice, who can then enter the access key 906 into a thin client web portal of a cloud-based system, confirming the validity of the access key 906 to said professional or medical provider. In some embodiments, the patient can subsequently be booked for a clinic visit, and further patient information intake and protection can be at the discretion of professional or medical user's practice and staff. According to some embodiments, randomly generated clinical engagement keys can be deleted from the system once validated, and/or can be invalid after a certain time frame.

In some embodiments, studies stored in cloud-based library (prior uploaded studies 306 of FIG. 3) can be anonymous and imaging metadata can contain non-identifying user ID. Referring to FIG. 2, according to some embodiments, as it pertains to privacy and security of uploaded imaging data, some embodiments comprising cloud-based imaging systems can contain an access control system 204, and various private information protection mechanisms such as anonymizing server application 201 (of anonymous data storage) to de-identify uploaded medical imaging studies. For example, similar to the disclosure in International Patent Application No. WO2013123085A1, an anonymization template comprises a plurality of DICOM entries, each entry corresponding to a data item of a medical image to be anonymized, via deletion of the data entry with the option for replacement with an anonymous consumer user ID.

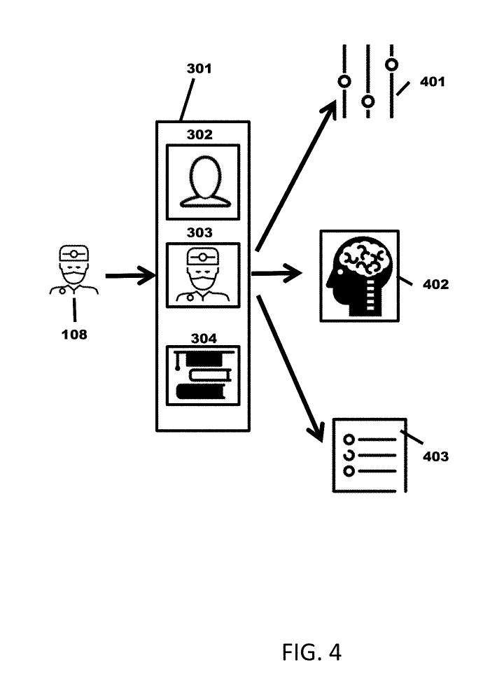

Some embodiments include professional or medical user account creation. For example, referring to FIGS. 4, 5 and 14, in some embodiments, a new professional or medical user can create an account for a cloud-based system access via sign up portal 1401. In some embodiments, this access can be granted free of payment to professional or medical service providers or users. In some embodiments, a professional or medical user data collection system can collect professional or medical practice data such as but not limited to: name, address, type of professional or medical degree, educational and clinical training history, professional or medical certifications, subspecialty certifications, copy of institutional/hospital credentials of said provider's scope of practice, background check, medical license information, and clinical interests within scope of practice. In some embodiments, the professional or medical practice data can be used in a matching of the professional or medical user and a consumer user (such as consumer user 101).

In some embodiments, upon completion of a new application by professional or medical user 108 in professional or medical user sign up portal 1401, a professional or medical user 108 can be informed that an application submitted can be cross-referenced and validated prior to approving creation of the account. In some embodiments, professional or medical service providers or users 108 must agree to provided information cross referencing and validation, including a possible background check. In some embodiments, professional or medical service providers or users' credential verification and background check can be processed automatically or manually by servers and staff of embodiment's cloud-based system or can be processed by third party provider.

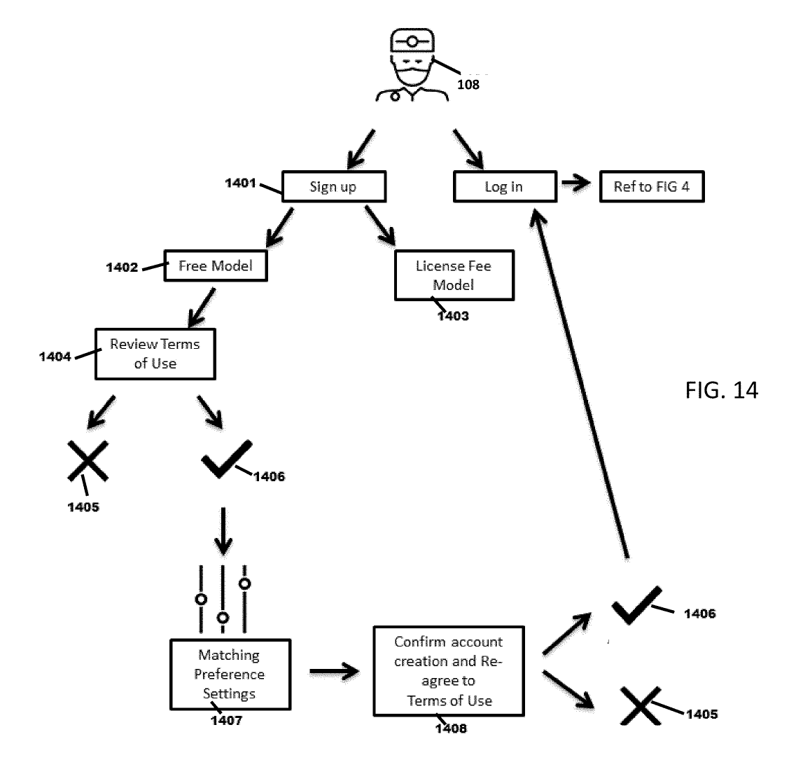

Referring to FIG. 14, in some embodiments, upon successful verification of a professional or medical user's credentials, the account can be approved and a professional or medical user 108 is respectively notified that account is ready for activation. In some embodiments, sign-up 1401 can be for a license fee model 1403 or a free model 1402. In some embodiments, prior to successful activation of a professional or medical account, professional or medical service providers or users 108 must accept a license and terms of use agreement 1404. In some embodiments, successful acceptance of the license and terms of use agreement (shown as 1406) can grant professional or medical service providers or users access to professional or medical workspace platform (represented as 303 in FIG. 4) and can proceed to match preference settings 1407 and/or confirm account creation and re-agree 1408. In some embodiments, declining professional or medical license and terms of use agreement 1405 can result in deletion of all data stored within cloud-based system servers in association with failed account creation event. In some embodiments, the aforementioned professional or medical license and terms of the use agreement can include the following provisions and terms:

1. The cloud-based service is meant only as an anonymous screening method to efficiently and widely direct healthcare consumers to professional or medical experts. Therefore, professional or medical service providers or users cannot provide any diagnostic or therapeutic treatment advice or plans within the present cloud-based system. The professional or medical user is reminded that diagnostic workup, and treatment options should be discussed during direct personal consumer and professional or medical user clinical engagements.

2. The user's expressed understanding that the use of one or more embodiments of the invention does not guarantee a time frame for a positive match between a consumer and professional or medical user. Furthermore, the use of one or more embodiments of the invention does not make any claims or guidelines about the timeframe in which the direct engagement of consumer and professional or medical user can occur following a positive match identified by the system.

3. The user's expressed understanding that the cloud-based system can intentionally reduce uploaded, transmitted, processed, and stored file sizes by “smart data cropping”, in reference to the embodiment's application that functions to upload, transmit, process, and/or store certain parts of a study. Certain parts of the study (e.g., certain images) refers to those images that are clinically relevant to consumer user's self-reported condition.

4. The automatic systems' smart data cropping application function is programmed based on experience of clinical best practices, and thereby may not represent the images that other clinicians would deem clinically relevant or irrelevant for a particular condition.

5. Table 1 outlines a set of rules governing smart data cropping functions that can be reprogrammed at any time to manage and maintain the “smart data cropping” application.

6. Professional or medical service providers or users are not permitted to freely communicate with consumer users through the system with the exception of the communication mechanisms provided within the cloud-based system clinical engagement platforms as illustrated in FIGS. 10A-10B and 11.

7. There is no identifying consumer user information maintained within cloud-based system. All uploaded studies are anonymized upon immediate access to the cloud-based system.

8. Personal consumer communication mediums or handles are not collected or stored by system. In other words, identifying communication handles such as but not limited to name, physical address, phone number(s), messaging handles, e-mail address are not collected.

9. Accounts where login information is not available to professional or medical user for whatever reason are deemed compromised.

10. Compromised professional or medical user accounts can have all associated matched patient studies and respective match history deleted. The account will not be deleted and can be re-instated following deletion of anonymous consumer user content associated with account.

In some embodiments, professional or medical user account access to patient platform 303 can require payment or licensing. In some embodiments, professional or medical service providers or user's licensing can be implemented at an individual user level and can be structured in payment models such as in a monthly subscription, and/or semi-annual subscription, and/or annual subscription, and/or number of consumer user matches, and/or number of consumer users engaged. In some embodiments, professional or medical user groups, practices, or institutions can engage in similar aforementioned licensing payment models, taking into account the number of professional or medical service providers or users within said group, practice, or institution that request access to the invention.

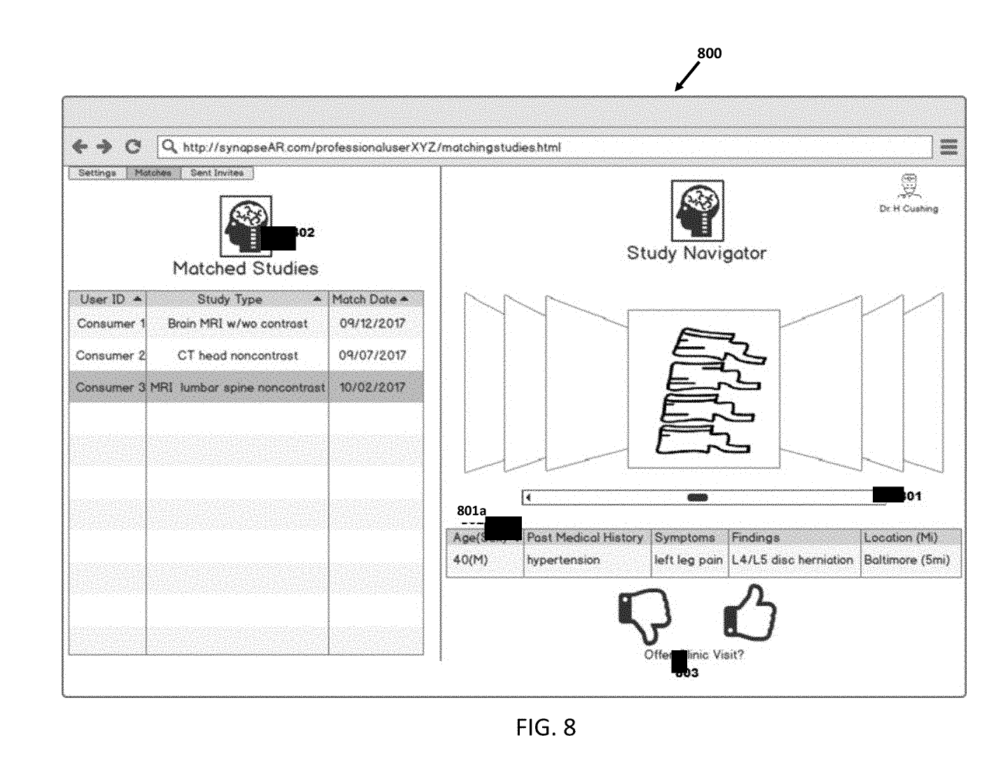

FIGS. 4, 7, 8 (GUI 800), and 10B illustrate professional or medical user navigation of provider portal. Referring to FIG. 4, according to some embodiments, choosing menu option “provider” (303 of FIG. 3) can present a professional or medical user with a host of options including but not limited to configuring matching preferences 401, navigation of prior matched studies (402 of FIGS. 4 and 8), access to literature 304, and matched consumer user engagement (403 of FIG. 4). According to some embodiments, professional or medical user selection of configuration of matching preferences 401 option can permit a provider to customize potential consumer user matches according to options such as, but not limited to, practice location, geographical range for matching consumer users, and clinical interests. FIG. 7 is an illustration of professional or medical user workstation with GUI 700 that can be used to enter and select matching preferences in a desktop version according to some embodiments of the invention.

FIG. 6 depicts a GUI 600 of a consumer user workstation showing downloads 610 of imaging studies in native or post processing modalities including 3D volume renditions 620 in accordance with some embodiments of the invention. FIG. 8 is an illustration of professional or medical user workstation for selecting and viewing anonymous user imaging studies in native or post processing modalities in a desktop version according to some embodiments of the invention. In some embodiments, the GUI 800 includes a professional or medical user selection of matching studies menu option 402 that can direct a professional or medical user to review a list of anonymous consumer user imaging studies that match professional or medical user's preferences entered in professional or medical user preferences menu option 401 (FIG. 4). In some embodiments, a professional or medical user can organize said list according different criteria such as, but not limited to, consumer user: age, sex, location, past medical history, and self-reported diagnosis. In some embodiments, selecting matching studies menu option 402 can allow a professional or medical user to engage consumer user 101. In some embodiments, the latter decision to engage consumer user 101 can be based on professional or medical user's assessment of consumer user 101 self-reported demographic data (i.e., through the use of a thumbs-up or thumbs down icon 803) and accompanying anonymous imaging studies 801 and patient data 801a.

With respect to access control to view anonymous imaging studies through the matching consumer user studies 402 menu option, conventional cloud-based systems have access control systems that define client access. For example, certain clients may or may not have access to certain medical data or processing resources based upon their respective access privileges, and the access privileges are configured based on particular institutional or facility policies (as discussed earlier). In some embodiments of the invention, access control system 204 (FIG. 2) does not grant system wide medical image data access, processing, or editing rights in association with professional or medical user role and credentials. Rather, in some embodiments, the access control system 204 can grant rights on an individual study basis. Therefore, in some embodiments, professional or medical service providers or users can be granted access to view only the studies they are specifically matched to. The purpose of the latter is to protect consumer user privacy and to facilitate a targeted and efficient medical imaging exchange between a consumer and professional or medical service providers or users based on a matching algorithm executed by user matching system 205. Hence, in some embodiments, professional or medical service providers or users can be granted viewing and imaging processing rights by the access control system 204 when a set of demographics, practice scope, and individual practice preference criteria of professional or medical user directly match that of data inputted by the consumer and as processed by the automated function of user matching system 205, contained within server 106 (FIG. 1 and FIG. 11). As depicted in FIG. 2, in some embodiments, this can include 3D volume rendering 202 from one or more anonymous data storage 201 of a networked system 203. In some embodiments of the invention, the networked system 203 can access literature 206, and transmit updates to a mobile phone 208a, computer system 208b, tablet or personal digital assistant 208c, and/or wearable computer system 208d.

Referring to FIG. 10A, in some embodiments, a professional user 108 can review sent clinical engagement invitations and view anonymous imaging studies through the matching consumer user studies 402 menu option and decline 802a or offer 802b a clinical engagement 802. If accepting a clinical engagement, the system can send an invitation for clinical engagement and access key is sent to the customer 1005. According to some embodiments of the invention, a professional or medical user selection of clinical engagements menu option 403 (matched consumer user engagement 403 of FIG. 4) can direct a professional or medical user to review a list of sent clinical engagement invitations. In some embodiments, as discussed above, clinical engagement invitations can be sent as a randomly generated access keys generated by random key generator 1302. In some embodiments, the access key can be used by consumer user when booking an appointment by phone, email, or electronic interface with professional or medical user's place of practice. Referring to the display 1050 of FIG. 10B, in some embodiments, professional or medical service providers or users 108 can be enabled to see a list of clinical engagements they have sent to anonymous consumer users (sent invitations 403), and whether that user has booked a clinic appointment with the clinical engagement invitation access key (list 1007). In some embodiments, professional or medical service providers or users 108 can be enabled to organize said list 1007 according different criteria such as, but not limited to, consumer user: age, sex, location, past medical history, and self-reported diagnosis. In some embodiments, clinical engagement invitation keys can expire after a certain time frame. According to some embodiments, professional or medical service providers or users can generate new keys for consumer users who have allowed their clinical engagement access key to expire. Accordingly, in some embodiments, consumer users can have an option to request a new clinical engagement key if the originally generated key has expired. In some embodiments, the display 1050 can include a medical image 1002 of a selected patient and/or use (shown as user ID 1, marked as 1001). Further, in some embodiments, the display 1050 can include a patient list 1003 that can include age, and/or past medical history, and/or symptoms, and/or findings, and/or location information.

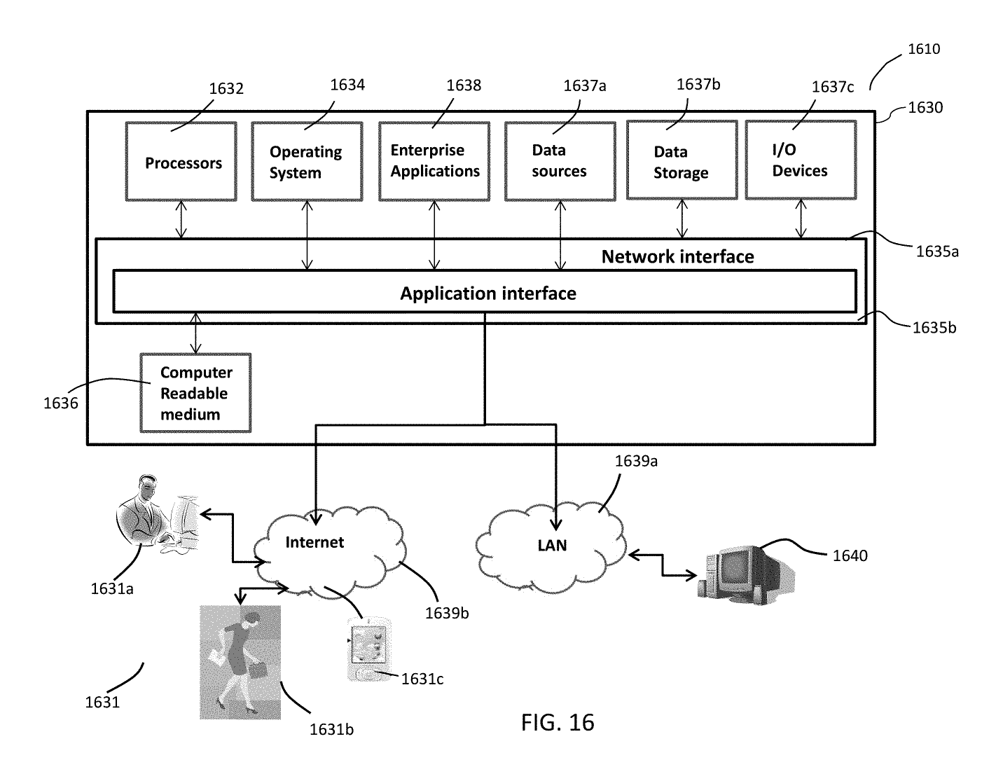

FIG. 16 illustrates a computer system 1610 configured for operating and processing components of the system and methods described herein, including the systems or methods of FIGS. 1-8, 9A-9B, 10A-10B, and 11-15. In some embodiments, the computer system 1610 can operate and/or process computer-executable code of one or more software modules of the system and method described herein. Further, in some embodiments, the computer system 1610 can operate and/or display information related to one or more graphical user interfaces. In some embodiments, the system 1610 can comprise at least one computing device 1630 including at least one processor 1632. In some embodiments, the at least one processor 1632 can include a processor residing in, or coupled to, one or more server platforms. In some embodiments, the system 1610 can include a network interface 1635a and an application interface 1635b coupled to the least one processor 1632 capable of processing at least one operating system 1634. Further, in some embodiments, the interfaces 1635a, 1635b coupled to at least one processor 1632 can be configured to process one or more of the software modules (e.g., such as enterprise applications 1638). In some embodiments, the software modules 1638 can include server-based software modules. In some embodiments, the software modules 1638 can operate to host at least one user account and/or at least one client account, and operating to transfer data between one or more of these accounts using the at least one processor 1632.

With the above embodiments in mind, it should be understood that the invention can employ various computer-implemented operations involving data stored in computer systems. Moreover, the above-described databases and models throughout the system can store analytical models and other data on computer-readable storage media within the system 1610 and on computer-readable storage media coupled to the system 1610. In addition, the above-described applications of the system can be stored on computer-readable storage media within the system 1610 and on computer-readable storage media coupled to the system 1610. These operations are those requiring physical manipulation of physical quantities. Usually, though not necessarily, these quantities take the form of electrical, electromagnetic, or magnetic signals, optical or magneto-optical form capable of being stored, transferred, combined, compared and otherwise manipulated.

In some embodiments of the invention, the system 1610 can comprise at least one computer readable medium 1636 coupled to at least one data source 1637a, and/or at least one data storage device 1637b, and/or at least one input/output device 1637c. In some embodiments, the invention can be embodied as computer readable code on a computer readable medium 1636. In some embodiments, the computer readable medium 1636 can be any data storage device that can store data, which can thereafter be read by a computer system (such as the system 1610). In some embodiments, the computer readable medium 1636 can be any physical or material medium that can be used to tangibly store the desired information or data or logic instructions and which can be accessed by a computer or processor 1632. In some embodiments, the computer readable medium 1636 can include hard drives, network attached storage (NAS), read-only memory, random-access memory, FLASH based memory, CD-ROMs, CD-Rs, CD-RWs, DVDs, magnetic tapes, other optical and non-optical data storage devices. In some embodiments, various other forms of computer-readable media 1636 can transmit or carry logic instructions to a computer 1640 and/or at least one user 1631, including a router, private or public network, or other transmission device or channel, both wired and wireless. In some embodiments, the software modules 1638 can be configured to send and receive data from a database (e.g., from a computer readable medium 1636 including data sources 1637a and data storage 1637b that can comprise a database), and data can be received by the software modules 1638 from at least one other source. In some embodiments, at least one of the software modules 1638 can be configured within the system to output data to at least one user 1631 via at least one graphical user interface rendered on at least one digital display (e.g., to a display of mobile computing device 1631c).

In some embodiments of the invention, the computer readable medium 1636 can be distributed over a conventional computer network via the network interface 1635a where the system embodied by the computer readable code can be stored and executed in a distributed fashion. For example, in some embodiments, one or more components of the system 1610 can be coupled to send and/or receive data through a local area network (“LAN”) 1639a and/or an internet coupled network 1639b (e.g., such as a wireless internet). In some further embodiments, the networks 1639a, 1639b can include wide area networks (“WAN”), direct connections (e.g., through a universal serial bus port), or other forms of computer-readable media 1636, or any combination thereof.

In some embodiments, components of the networks 1639a, 1639b can include any number of user devices such as personal computers including for example desktop computers, and/or laptop computers, or any fixed, generally non-mobile internet appliances coupled through the LAN 1639a. For example, some embodiments include personal computers 1640a coupled through the LAN 1639a that can be configured for any type of user including an administrator. Other embodiments can include personal computers coupled through network 1639b. In some further embodiments, one or more components of the system 1610 can be coupled to send or receive data through an internet network (e.g., such as network 1639b). For example, some embodiments include at least one user 1631 coupled wirelessly and accessing one or more software modules of the system including at least one enterprise application 1638 via an input and output (“I/O”) device 1637c. In some other embodiments, the system 1610 can enable at least one user 1631 to be coupled to access enterprise applications 1638 via an I/O device 1637c through LAN 1639a. In some embodiments, the user 1631 can comprise a user 1631a coupled to the system 1610 using a desktop computer, and/or laptop computers, or any fixed, generally non-mobile internet appliances coupled through the internet 1639b. In some further embodiments, the user 1631 can comprise a mobile user 1631b coupled to the system 1610. In some embodiments, the user 163 lb can use any mobile computing device 1631c to wirelessly couple to the system 1610, including, but not limited to, personal digital assistants, and/or cellular phones, mobile phones, or smart phones, and/or pagers, and/or digital tablets, and/or fixed or mobile internet appliances.

In some embodiments of the invention, the system 1610 can enable one or more users 1631 coupled to receive, analyze, input, modify, create and send data to and from the system 1610, including to and from one or more enterprise applications 1638 running on the system 1610. In some embodiments, at least one software application 1638 running on one or more processors 1632 can be configured to be coupled for communication over networks 1639a, 1639b through the internet 1639b. In some embodiments, one or more wired or wirelessly coupled components of the network 1639a, 1639b can include one or more resources for data storage. For example, in some embodiments, this can include any other form of computer readable media in addition to the computer readable media 1636 for storing information, and can include any form of computer readable media for communicating information from one electronic device to another electronic device.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g., a cloud of computing resources.

The embodiments of the invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods that can be processed by one or more machines or processors that can be connected over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable logic instructions, data structures, program modules or other data.

Although method operations can be described in a specific order, it should be understood that other housekeeping operations can be performed in between operations, or operations can be adjusted so that they occur at slightly different times, or can be distributed in a system which allows the occurrence of the processing operations at various intervals associated with the processing, as long as the processing of the overlay operations are performed in the desired way.

Certain portions of the descriptions herein are presented in symbolic representation and at times in terms of algorithms that represent processing of data bits by a computer processor and memory. Algorithmic representations and detailed descriptions are presented in the format employed by those expert in the art of medical data acquisition, storage, transmission, and processing, to facilitate understanding to the invention by those skilled in the art. The term algorithm is utilized within embodiments to represent a self-consistent operation sequence producing a requested result. Similarly, labels applied to describe embodiments of the invention were applied for convenience of discussion and description of the various embodiments of the invention.

In the above discussion, all described embodiments of the invention are presented as possible embodiments of the invention. Physical assembly and execution of the invention can contain a number of embodiment variants; however, the latter shall not depart from the broad spirit of the description and embodiments as described. Similarly, all figures, illustrations, and drawings are created for convenience of conveying certain embodiments of the invention and thus are not to be interpreted as restrictive in the realization and/or execution of the invention. Further, it will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

1. A server comprising:
a processor coupled to a server storage configured to be coupled to a network; and
a non-transitory computer readable memory in communication with the processor and storing logic instructions that, when executed by the processor, cause the server to:
operate a gateway manager coupled to the server storage over the network, the gateway manager establishing a data transfer between at least one user and the server storage based on an access right;
process a request for data from an application running at a networked device associated with the user;
act on a request from the user to upload or download medical data to or from the server storage;
act on a request from the user to access and apply one or more process tools operating on at least a portion of the medical data stored on the server storage according to input from a user; and
provide a rule-based matching between two or more users based on at least a portion of the medical data and at least one rule, wherein at least some medical data associated with a first user is made accessible to at least one other user based on the at least one rule.

2. The server of claim 1, further comprising the non-transitory computer readable memory storing logic instructions that, when executed by the processor, cause the server to anonymize at least a portion of the medical data uploaded to the server storage.

3. The server of claim 2, wherein the server anonymizing at least a portion of the medical data automatically relabels the at least a portion of the medical data with non-identifying user characteristics.

4. The server of claim 1, further comprising the non-transitory computer readable memory storing logic instructions that, when executed by the processor, cause the server to image process at least a portion of the medical data using the one or more process tools operating on at least a portion of the medical data stored on the server storage.

5. The server of claim 4, wherein the image processing comprises at least one of rescaling, margin cropping, compression, file size reduction, processing metadata to a searchable list and/or index, encryption, modality conversion, and sharing of medical data between two or more users based on the at least one rule.

6. The server of claim 1, wherein the medical data comprises medical image data.

7. The server of claim 6, wherein the medical image data comprises radiological study data.

8. The server of claim 1, wherein the access right is governed by a license.

9. The server of claim 1, wherein the access right is license-free.

10. The server of claim 1, wherein the server storage is a cloud-based server storage coupled to the network.

11. The server of claim 1, wherein the at least one other user is a medical practitioner or healthcare service provider and the first user is a patient or potential patient of the at least one other user.

12. The server of claim 1, wherein the at least one rule comprises a preference of the at least one other user.

13. The server of claim 12, wherein the at least one rule comprises a preference of the at least one other user selected from one or more parameters or inputs on a display of the system that is coupled to the server storage.

14. The server of claim 1, wherein the at least one rule comprises the type of professional or medical degree of the at least one other user, educational and clinical training history of the at least one other user, professional or medical certifications of the at least one other user, subspecialty certifications of the at least one other user, institutional or hospital credentials of the at least one other user's scope of practice, a background check of the at least one other user, medical license information of the at least one other user, and clinical interests within scope of practice of the at least one other user.

15. The server of claim 13, wherein the one or more parameters or inputs comprises at least one of a diagnosis, a self-reported diagnosis, a study type, insurance status, geographic distance between the first user and the at least one other user, the age of the first user, and study date.

16. The server of claim 1, wherein the non-transitory computer readable memory storing logic instructions that, when executed by the processor, cause the server to analyze clinical relevancy of at least a portion of the medical data using the one or more process tools operating on at least a portion of the medical data stored on the server storage, the server identifying any non-clinically relevant medical data and any clinically relevant medical data based on one or more internal rules; and further configured to optionally discard non-relevant medical data.

17. The server of claim 1, wherein the non-transitory computer readable memory storing logic instructions that, when executed by the processor, cause the server to match at least some of the medical data based on a diagnosis defined by at least one of the users and/or extracted from metadata from the medical data or from data associated with stored medical data.

18. The server of claim 1, wherein the non-transitory computer readable memory storing logic instructions that, when executed by the processor, cause the server to upload the medical data directly from an institution or facility where the medical data was acquired using an upload link and temporary access key provided by a user.

19. The server of claim 1, wherein the non-transitory computer readable memory storing logic instructions that, when executed by the processor, cause the server to enable the first user to view engagements offered by the at least one other user after the at least one other user has reviewed at least some of the medical data associated with the first user.

20. The server of claim 19, wherein the non-transitory computer readable memory storing logic instructions that, when executed by the processor, cause the server to enable the first user to receive an access key for an engagement offered by the at least one other user after the at least one other user has reviewed at least some of the medical data associated with the first user, and further to enable the first user to book an appointment with the at least one other user through the server using the access key.
